(19) United States
(12) Patent Application Publication
(10) Pub. No.: US 2025/0208107 A1
(43) Pub. Date: Jun. 26, 2025

(54) PLANT STALK STRENGTH MEASURING DEVICE

(71) Applicant: Syngenta Crop Protection AG, Basel (CH)

(72) Inventors: Soumitra Khair, Downers Grove, IL (US); Miao Liu, Downers Grove, IL (US); Scott Tragesser, Monroeville, IN (US); Joao Oliveira, Minas Gerais (BR)

(73) Assignee: Syngenta Crop Protection AG, Basel (CH)

(21) Appl. No.: 18/847,843

(22) PCT Filed: Mar. 13, 2023
(86) PCT No.: PCT/US23/15085

§ 371 Date: Sep. 17, 2024

Related U.S. Application Data
(60) Provisional application No. 63/321,346, filed on Mar. 18, 2022.

(51) Int. Cl. G01N 33/00; A01D 75/00; A01H 1/04; A01H 6/46; G01L 5/00; G01N 3/20

(52) U.S. Cl. CPC G01N 33/0098; A01D 75/00; A01H 1/04; A01H 6/4684; G01L 5/0061; G01N 3/20

(57) ABSTRACT

An apparatus for measuring stalk strength of a plant is disclosed. A force sensor is mounted to a harvester in a position to measure the resistance to crushing of the plant stalk by a stalk roll of the harvester. The apparatus may include a pair of counter rotating stalk rolls that pull and crush the plant stalk between them, a force sensor coupled to the stalk rolls for measuring a force exerted on the roller by the plant stalk in resistance to crushing by the stalk roll, and a temperature sensor coupled to the roller for measuring a temperature at or around the force sensor. The output of the force sensor is corrected for temperature-based drifts based on the output of the temperature sensor. Data collected by the invention can be advantageously used in a breeding program wherein breeding decisions are made based at least in part on stalk strength.

### RELATED APPLICATIONS

This application claims priority to U.S. Provisional Patent Application No. 63/321,346 filed 18 Mar. 2022, the contents of which are incorporated by reference herein in its entirety.

### FIELD OF THE INVENTION

The present invention relates generally to apparatus and methods for evaluating the strength of a plant stalk, such as a corn plant in a plot.

### BACKGROUND OF THE INVENTION

The invention relates generally to crop harvesters and, more specifically, to a corn stalk strength measuring device mounted on a harvester for harvesting corn and methods for measuring corn stalk strength.

Stalk lodging in corn is the breakage of the corn stalk below the ear. Stalk lodging in corn results in increased harvest losses, slower harvest equipment speeds, increased drying cost and in most cases, a significant volunteer corn problem the following season. Yield losses from stalk lodging range from 5 to 25 percent nationwide. Stalk lodging is typically caused by one or more of the following: late season severe weather, damage to the stalk by a pathogen, and stalk rot disease. Resistance to root and stalk lodging are some of the most important traits selected for in commercial maize breeding.

Example scenarios leading to corn stalk lodging include the following. Plant population levels that are too high decrease the amount of light in the crop canopy and cause the corn plants to become tall and thin. The physical strength of the corn stalk under these conditions is significantly reduced. In addition, plant-to-plant competition for light, nutrients, and water enhances the competition for carbohydrates between the stalk and ear within the plant, thus reducing the vigor of the cells in the stalk and predisposing them to invasion by stalk rot. Extremes in soil moisture can increase the occurrence of stalk lodging, such as can occur due to late season severe precipitation. Excessive soil moisture retards root growth and development, leading to a less than optimum root system which cannot adequately support plant growth. On the other hand, drought-like conditions stress the crop and enhance the development of stalk rot by reducing movement of sugars to the root system. Nutrient imbalances and/or deficiencies predispose corn plants to stalk rot and stalk lodging. For example, high nitrogen fertility levels coupled with low potassium levels enhance the potential for stalk rot. High nitrogen levels enhance lush vegetative growth, while low potassium levels increase the amount of premature stalk death.

Together, these conditions produce an ideal situation for stalk rot and lodging. Conversely, low levels of soil nitrogen may result in less vigorous plants which put all their available energy into producing grain. This leaves the stalk vulnerable to stalk rot organisms and, ultimately, stalk lodging. Damage caused by the corn rootworm and the European corn borer can predispose the corn plant to invasion by stalk rotting organisms, as well as lead to outright yield loss. Corn rootworm larvae decrease the amount of water and nutrient uptake by feeding on the roots, whereas the European corn borer damages the stalk by feeding on the pith and the vascular tissue. In either situation, the corn plant is placed under physiological stress, which favors both stalk rot development and stalk lodging. These insects can also encourage the development of stalk rots by reducing the photosynthetic area of the plant, causing wounds through which pathogens enter stalks and roots, and carrying disease inoculum into tissues. Cultural practices which increase the amount of disease or insect pressure can also increase the amount of lodging that occurs in the corn crop. Leaving disease-infected corn stubble on the soil surface through reduced tillage methods can increase the incidence of stalk rot and stalk lodging in monoculture. Continuous cropping of corn also enhances the potential for insect problems such as European corn borer and corn rootworm.

One of the approaches to preventing stalk lodging is the development of commercial hybrid seed varieties with improved stalk strength. Currently, corn development programs commonly include selection of new corn varieties for advancement based at least in part on stalk strength. Stalk strength measurements can be taken of plants of the corn varieties at various times throughout the growing season, however, the most common practice is to count or estimate broken plants prior to harvest. Good expression of the stalk strength trait depends upon winds that are strong enough to break weak plants yet not so severe as to cause widespread, indiscriminate lodging on all plots.

Various types of apparatus have been developed which measure susceptibility of a corn stalk to lodging. One example approach is shown by Mann et al. in U.S. Pat. No. 7,987,735B2. Therein, a test device comprising an accelerometer is coupled to a plant stalk and then a test force is manually applied. The resulting stalk vibrations are measured by the accelerometer and used to estimate the plant's susceptibility to lodging. However, the inventors herein have recognized that in such an approach, since a person must walk through the plot of growing corn, conduct the measurement manually on the plant using the hand-held test device and record the measurement for the plant, it is a time-consuming and labor-intensive process that can only be performed on a relatively small number of plants on a given plot, and furthermore may not be feasible for larger plots.

Another example approach is disclosed by Deppermann et al. in U.S. Pat. No. 7,401,528B2 wherein an apparatus with a conveyor-driven pulling finger pulls a plant stalk and a force sensor of the apparatus measures a resistive force encountered in response to the pulling. However, the inventors herein have recognized various issues with such an approach. As one example, the machinery involved in operating the apparatus may cause temperature-driven variations in the output of the sensor, resulting in inaccurate stalk strength estimation. As another example, the arrangement of the strain gauge sensors to form a Wheatstone bridge circuit renders the system sensitive to change in resistivity when force is introduced during pulling. Particularly, since the sensors are arranged at different heights, there may be unequal force exerted on individual sensors, causing an improper force to be read.

The inventors herein have further recognized that currently available approaches, including those discussed above, may not provide a statistically accurate measurement of the stalk strength of plants in a plot. For example, due to the time and labor-intensity involved, not all plants of a large plot may be assessed. Typically, only a subset of all plants of a plot are assessed, and the results extrapolated to the remainder of the plot. However, if these plants had growth issues, genetic issues, or were otherwise statistical outliers, the stalk strength results of the plot may be skewed. Further biases may be introduced during the subjective selection of plants for testing by the tester, and/or based on plot density estimations. Since stalk strength is relied on to select plants for breeding programs, errors may be unintentionally introduced. There is a need, accordingly, for an automated apparatus and improved methods for taking stalk strength measurements for a statistically significant number of plants on a large number of plots.

### SUMMARY OF THE INVENTION

The invention consists of a stalk strength measuring device mounted on a corn harvester for measuring the strength of stalks of a variety of corn as they are being harvested by the harvester. In particular, the device is able to automatically take stalk strength measurements for a statistically significant number of plants of a plot, allowing for a more accurate estimate of the stalk strength of plants in the plot. As non-limiting embodiments, the device may estimate the stalk strength of a statistically relevant number of plants of a plot (or row or other plot parameter), including but not limited to a higher than threshold percentage of plants of a plot (e.g., at least 50% of plants of the plot). In some embodiments, the device may estimate the stalk strength of each and every plant on the plot.

In a particular embodiment, the device is mounted on a corn harvesting combine head including a pair of counter-rotating stalk rolls. Gathering chains of the header pull the corn stalks toward and into the stalk rolls which engage the stalks and pull them in between the rolls, crushing the stalks in the process. One or more force or pressure sensors, such as one or more strain gauges, may be mounted on one or more or each of the stalk rolls in a relative configuration that optimizes accurate stalk strength estimation. In response to the stalk crushing process, the sensors/gauges provide an output signal proportional to the resistance to crushing of the stalk of each plant that passes through the stalk rolls. This signal is processed in a controller using digital signal processing to provide a numerical value representative of stalk strength of the corresponding plant. One example of a strain gauge sensor that can mounted on a combine head is disclosed in U.S. Pat. No. 8,215,191, the entire contents of which are incorporated by reference herein.

Further, the sampling frequency of the sensor(s) may be adjusted so that the number of plants sampled may be accurately determined by the signal peaks. For example, strain gauge output (e.g., strain gauge output peaks) may be overlayed with plot density maps to determine a plant stand count indicative of the number of plants in a plot that were processed for stalk strength measurements. The harvester may then be operated to potentially harvest each plant of the plot and receive signals indicative of the stalk strength of each plant of the plot. One example of a strain gauge sensor that can mounted on a combine head is disclosed in U.S. Pat. No. 8,215,191, the entire contents of which are incorporated by reference herein.

In further embodiments, the device may include geo-positional sensors/devices indicative of a position of the plant stalk being measured. By overlaying or correlating the output of the positional sensors with the output of the strain gauges, stalk strength may be mapped as a function of plant position within a plot.

In still further embodiments, the device may include a temperature sensor to account for temperature-based drifts in sensor output. Operation of heavy machinery such a combine may result in elevated temperatures in the vicinity of the strain gauges mounted on the combine harvesting head. The output of the strain gauge sensors may be adjusted with a correction factor based on the output of the temperature sensor.

In still further embodiments, the strain gauge sensors may be protected from environmental conditions through the presence of a sensor protection assembly that covers the sensors. The sensor protection assembly may include a sensor cover and a bracket for mounting the sensor cover to the stalk rollers. Further protection may be provided for underlying cables connecting the sensors to a controller.

The stalk strength measurements are recorded digitally and can be further analyzed for use in making decisions regarding use of the variety in a corn breeding program, such as for decisions involving use of the plant in breeding for stalk strength traits. Stalk quality is an important trait to farmers as they expect corn hybrids to stand until the crop is harvested. Any stalks that break below the ear can result in yield loss and economic loss. The traditional method of understanding stalk strength is to count broken stalks in research plots, but the plant breeding community also recognizes the limitations of this method: stalks may be weak for a variety of reasons (e.g., disease, lack of fertility, genetic weakness), but the stalk breakage may or may not occur in every environment. It would be desirable to count broken stalks, but the task is time and labor intensive for a trait that does not always express itself. The proposed stalk strength measurement captures a datapoint for every plot regardless of the mechanical failure of the plant(s). The relative strength/weakness of the stalks is captured regardless of whether the stalks have broken and the sheer number of datapoints collected by a combine gives a much better estimate of stalk strength over a larger number of environments. With this increased amount of data, plant breeders can make a better decision about the strength of the stalks for every hybrid. This leads to more confidence that the commercial hybrids sold to farmers will have high quality stalks and less potential yield loss. The invention may also be used to detect gaps of missing plants in a row of a plot of corn plants by the absence of a signal over a given distance which could be measured by a GPS device, radar, optical shaft encoders and the like associated with the invention.

It is further contemplated that the invention be used, again in association with a GPS device, radar, optical shaft encoders and the like to calculate a “fill ratio” representative of how uniformly corn plants of a given stalk strength are distributed in a plot harvested by a combine that includes the present invention.

In an alternative embodiment of the present invention, a laser beam is directed onto the stalk rolls, a sensor detects reflected laser light and changes in transit time is used to determine deflection of the stalk rolls in response to the crushing of stalks there between and therefore the resistance to crushing offered by the stalks.

In another alternative embodiment, a magnetic sensor, such as a Hall effect sensor, is mounted near the stalk rolls and measures deflection of the stalk rolls in response to the crushing of stalks therebetween and therefore the resistance to crushing offered by the stalks. Still other known force sensors may be relied upon.

In this way, a stalk strength measuring device is provided that provides a more accurate estimation of stalk strength of plants in a plot. By correlating force sensor output with plot maps, the number of plants assessed can be determined, allowing for a more statistically accurate estimate of the average stalk strength of the plot. Further, the stalk strength of each plant in the plot can be determined. By accounting for temperature-based sensor drifts that may occur due to heavy machinery operation, a more reliable stalk strength estimate of plants may be provided for use in breeding programs.

### DESCRIPTION OF THE FIGURES

FIG. 1 is front view of a harvester for corn used with an embodiment of the present invention.

FIG. 2 is an enlarged view of the harvester of FIG. 1 and showing gathering chains and stalk rolls used with an embodiment of the present invention.

FIG. 3 is an enlarged view of the gathering chains and stalk rolls of FIG. 2 from a lower perspective.

FIG. 4 is an enlarged view of the stalk rolls of FIGS. 2 and 3 from a lower perspective and with arrows indicating the respective counter rotation of the two stalk rolls.

FIG. 5 is an exploded perspective view of the mounting of the stalk rolls.

FIG. 6 is a view of the stalk roll drive shafts and drive shaft housing.

FIG. 7 is a perspective view of the drive shaft housing.

FIG. 8 is a plan view of bi-axial strain gauge installed on a drive shaft housing, coupled to leg sections of the housing.

FIG. 9 is a top view of another embodiment wherein the strain gauge sensors are installed at a base of the drive shaft housing, between the leg sections and at base of the brackets.

FIG. 10A is an enlarged view and FIG. 10B is a reduced view of a strain gauge mounted to the drive shaft housing in a recess.

FIG. 11 is a view of a terminal block mounted on the base of the drive shaft housing.

FIG. 12 is a circuit diagram of strain gauges base pad to connector cable according to an embodiment of the proposed invention.

FIG. 13 is a flow chart of Raw Signal Acquisition conversion to Digital Signal according to the disclosure.

FIG. 14 is a flow chart of the data acquisition and signal processing components of an embodiment of the proposed invention.

FIG. 15 is a flow chart of the data acquisition, signal processing and data storage components of an embodiment of the proposed invention for a plot in a field.

FIG. 16 is a view of a plot user interface of an embodiment of the proposed invention.

FIG. 17 is a diagrammatic view of ideal signal characteristics.

FIG. 18 is a diagrammatic view of an exemplary raw data signal generated by an embodiment of the present invention.

FIG. 19 is a diagrammatic view of the exemplary raw data signal generated by an embodiment of the present invention of FIG. 16 indicating the regions and features of FIG. 15.

FIG. 20 is a diagrammatic view of the exemplary raw data signal generated by an embodiment of the present invention of FIG. 16 and showing an overlay of low-pass filtered data.

FIG. 21 is a diagrammatic view of the low pass filtered data of FIG. 20.

FIG. 22 is a view of a simulation screen displaying the signal from position sensor and strain gauge sensor.

FIG. 23 is a block diagram illustrating coupling of the strain gauge sensors to an on-board controller, in accordance with an embodiment of the invention.

FIG. 24 compares a signal acquired from a strain gauge at a higher sampling frequency of 3000 Hz (Panel A) with asignal acquired from strain gauge at lower sampling frequency of 100 Hz (Panel B).

FIG. 25 is illustration of raw signal vs simulated signal representing peaks due to stalk crushing activity and plant location overlaid at bottom.

FIG. 26 is an example embodiment of a sensor protection assembly mounted on top of a strain gauge sensor positioned between the leg regions of the housing, such as to protect sensors mounted on a base of the drive shaft housing.

FIG. 27 is an example embodiment of a protection plate that may be mounted on the base of the drive shaft housing to hold the sensor protection assembly in place.

FIG. 28 is an example embodiment of a cable protection assembly that may be mounted on base pad the driving shaft to protect underlying cables coupling the sensors to the controller.

FIG. 29 is an example embodiment of a the housing mounted with the protection assembly components of FIGS. 26-28.

FIG. 30 is a high-level flow chart of an example method for stalk strength estimation using a stalk strength measuring device, in accordance with the present disclosure.

### DETAILED DESCRIPTION

A detailed description is provided for a stalk strength measuring device comprising one or more strain gauges, a temperature sensor, and a controller for processing the output of the sensors. The device can be mounted to a farming equipment navigating through a plot of plants. It will be appreciated that while the embodiments of the description illustrate the use of a stalk measuring device mounted on a harvester combine, this is not meant to be limiting and in further embodiments, the stalk measuring device can be coupled to other farming equipment without departing from the scope of the invention.

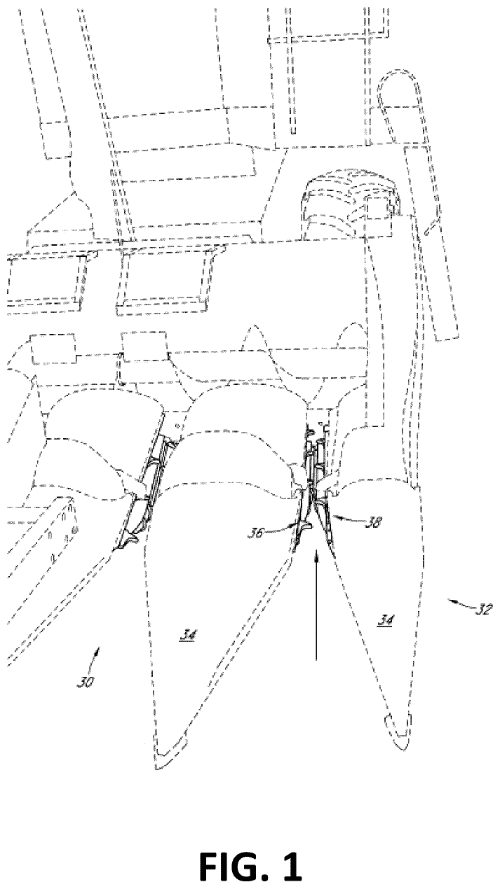

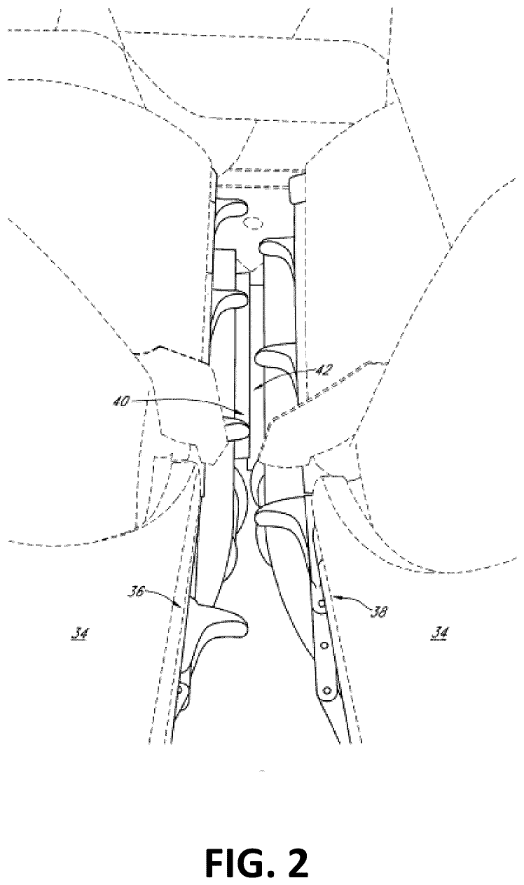

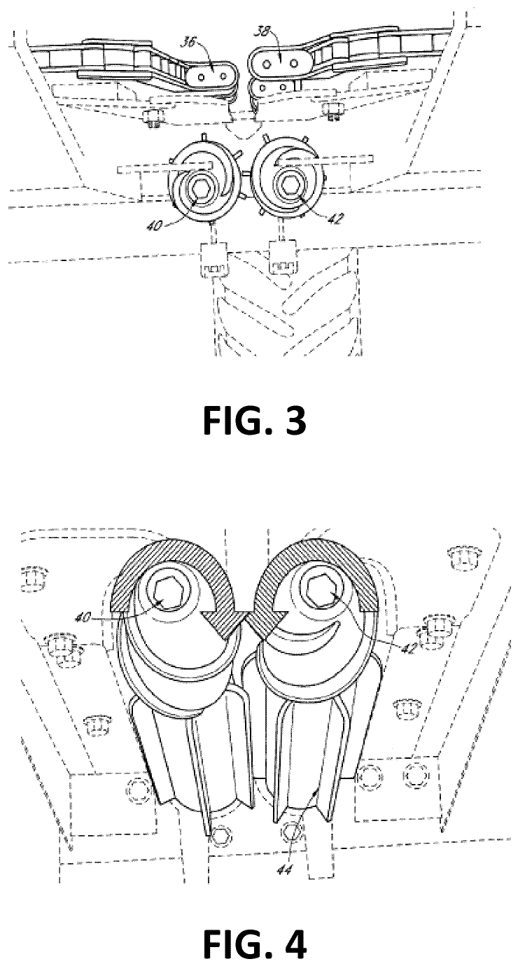

Referring to FIG. 1, a harvester, indicated generally at 30, is shown, including a header 32 and a plurality of dividers 34. Corn stalks being harvested by the harvester 30 pass between adjacent pairs of the dividers 34 and are engaged by stalk gathering chains 36 and 38 that assist in moving the stalks into the harvester 30. The V-shaped channels marked by the arrow is the area where the corn plants meet the gathering chains and are gathered in and pulled downward by rotating stalk rolls. In other embodiments, channels or grooves of other geometry may be provided for receiving the corn plants. The gathering chains guide the stalks toward and into contact with the pair of counter-rotating stalk rolls 40 and 42 (FIGS. 2-4). The intact stalks are engaged by the stalk rolls 40, 42 and pulled rapidly downward therebetween, being assisted by a series of blades 44 of the stalk rolls 40, 42. FIG. 4 shows a close-up view of the underside of the stalk rolls. Stalks are drawn in by the spiral shaped stalk roll tips and then drawn downward by the blades on the stalk rolls. Stalks are crushed between these stalk rolls as they are drawn downward and the amount of resistance to crushing is a measure of ultimate stalk strength.

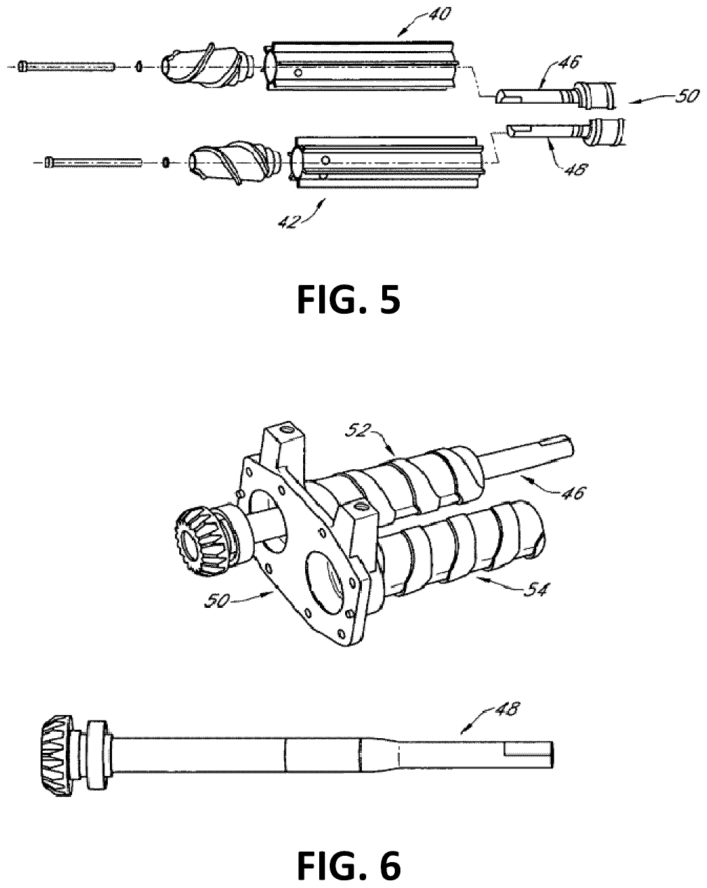

The stalk rolls 40, 42 are each rotated by a corresponding stalk roll drive shaft 46, 48 which themselves each rotate inside a stationary drive shaft housing 50, each journaled in a corresponding leg section 52, 54 of the housing 50 (FIGS. 5-7). The stalk rolls 40, 42 thus extend beyond the stationary drive shaft housing 50 but are functionally linked thereto by the drive shafts 46, 48 so that any non-longitudinal force exerted on the stalk rolls 40, 42 is transmitted to the stationary housing 50 via the drive shafts 46, 48. Accordingly, forces exerted on the stalk rolls 40, 42, can be measured by measuring the force on the stationary housing 50. More specifically, as the stalks are drawn through the stalk rolls 40, 42, the stalks are compressed by the stalk rolls 40, 42 that are spaced apart by a distance which will result in crushing of the stalks. The stalks, of course, resist crushing by an amount that is proportional to the stalk strength against crushing. This force is exerted on the stalk rolls 40, 42, in a direction tending to separate the stalk rolls 40, 42 or increase the distance between the stalk rolls 40, 42. Because of the transmission of this separation force to the drive housing 50 via the drive shafts 46, 48, a strain is placed on the drive housing 50 that is likewise in a direction tending to separate the housing leg sections 52, 54.

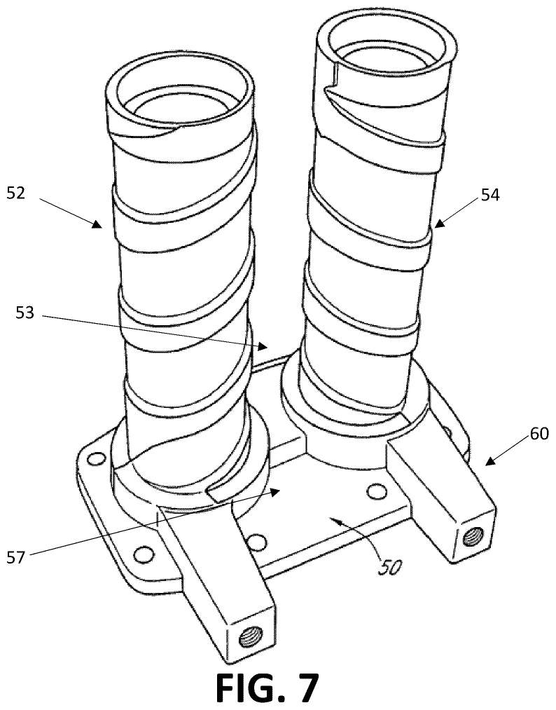

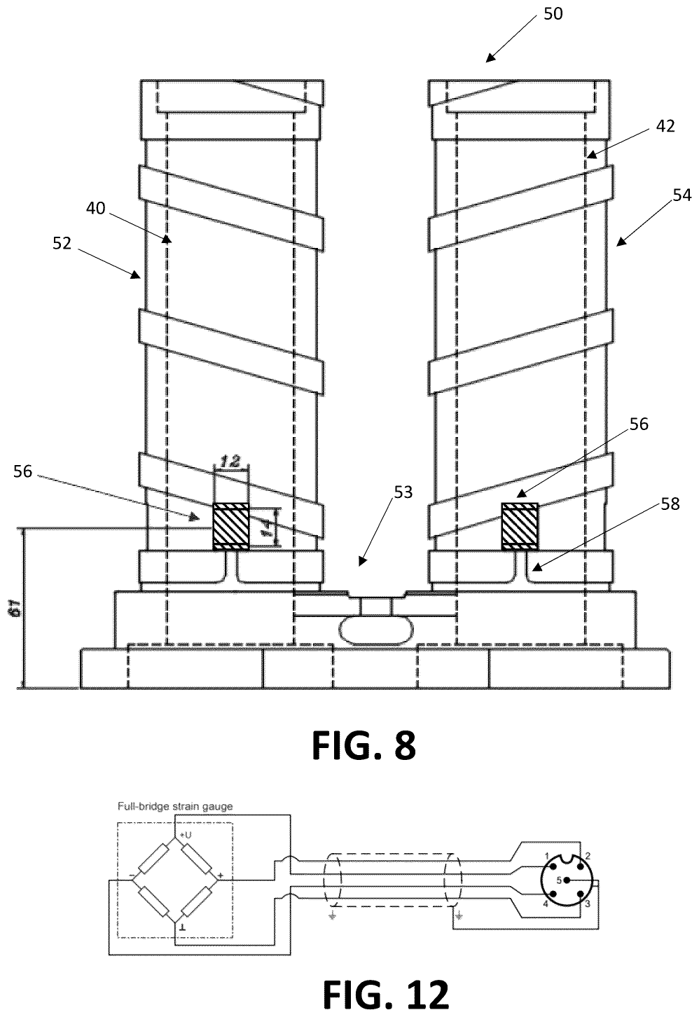

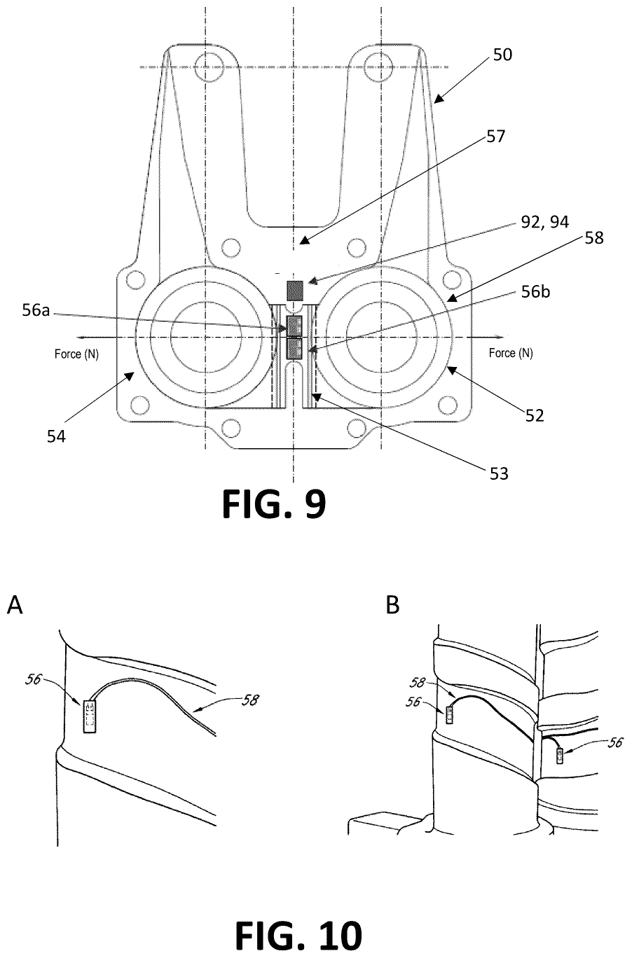

As shown in FIG. 7 for example, the housing 50 of the drive shaft assembly includes leg sections 52, 54 housing the corresponding drive shafts (46, 48, not shown in FIG. 7), a base region 57 and an inter-leg region 53. In embodiments, a strain sensor may be coupled to the housing in any location that allows for accurate strain estimation, such as in inter-leg region 53 (as shown in the embodiment of FIG. 9), or on corresponding leg regions 52, 54 (as shown in the embodiment of FIG. 8).

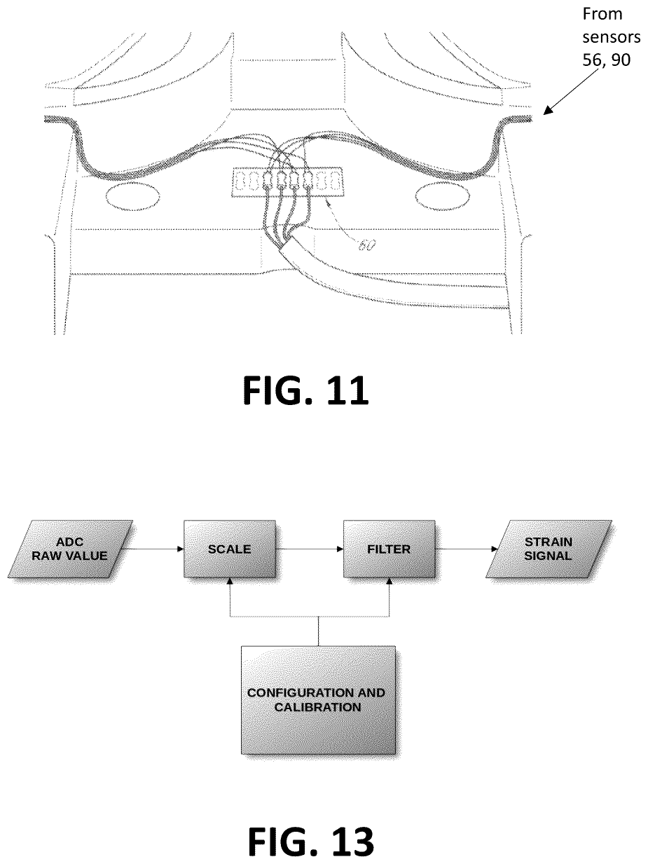

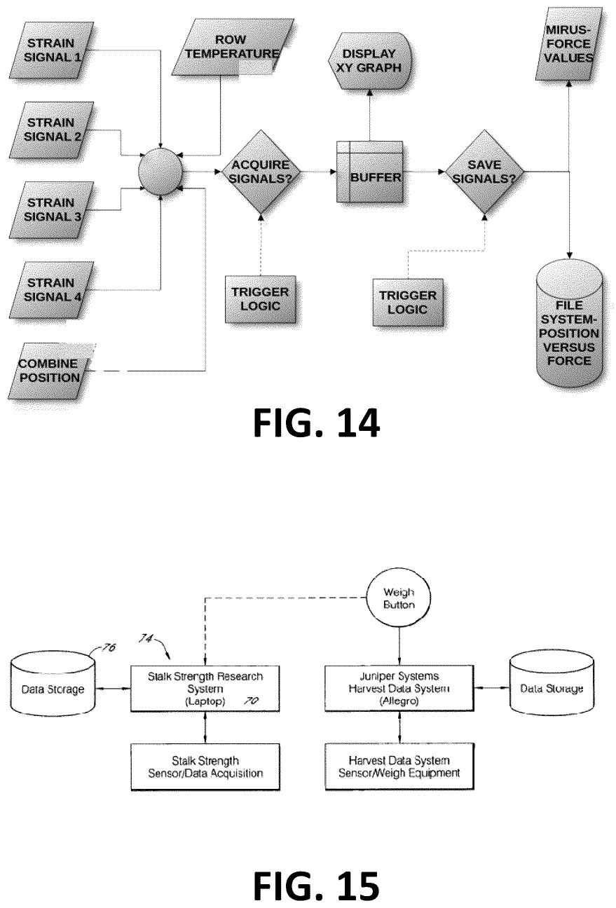

The strain exerted on the housing 50 is measured by one or more pressure or force sensors, depicted herein as one or more strain gauges 56. Any number of strain gauges may be provided mounted to the housing 50 in a configuration that enables compression or tension to be accurately sensed when a plant stalk is passed through the stalk rolls 40, 42. In one example embodiment, two strain gauges 56 (herein also referred to as 56a and 56b) are mounted in a bi-axial configuration (FIG. 8) to housing 50 to measure cross sectional force. In another embodiment, 2 double strain gauges 56a, 56b are mounted in the inter-leg region 53 at the base of the housing 50 to measure the compression at the base. This compression can be co-related with the strain for crushing the stalks. As shown in FIG. 7-8, the strain gauges 56a and 56b are mounted to points that experience compression or tension when force is applied during stalk crushing or harvesting. The strain gauges 56 can be mounted on the inside and/or outside surface of each of the housing leg sections 52, 54. In the harvester 30 of the disclosed embodiment, the housing leg sections 52, 54, have a raised spiral profile, leaving recesses or lands (FIGS. 7-8) and the strain gauges 56 are mounted in the recesses in the bi-axial configuration so as to decrease the likelihood of getting damaged due to contact with stalks and other debris that moves between and past the leg sections 52, 54. In another embodiment the strain gauge sensor can be installed in the base of the housing 50 (FIG. 9) so as to protect from wear and tear due to stalks during harvest and to read compression at center of housing 50 due to stalk crushing. In some embodiments, a mounting surface for each of the strain gauges may be provided by smoothing the surface of the leg section 52, 54 with a grinder or similar tool. Each of the one or more strain gauges 56 is attached to a corresponding mounting surface by any known method of sensor coupling including, but not limited to, application of epoxy or the like with a pair of wires 58 of each of the strain gauges 56 trailing through the recess toward the base of the housing 50, the wires also secured to the housing 50 by epoxy or the like. In embodiments, a terminal block 60 is mounted at the base of the housing 50 and the wires 58 are electrically connected to the terminal block 60 (FIGS. 10A, 10B and 11). The terminal box 60 further comprises a power source 90.

In addition to strain gauges 56, 56a, 56b, a temperature sensor 92 (depicted in FIG. 9) is also mounted to the housing 50. In embodiments, the temperature sensor may be placed adjacent the strain gauge sensors 546 of the depicted example embodiments of FIGS. 8 and 9. The inventors herein have recognized that operation of heavy machinery, such as the stalk crushing rolls of the harvester, can result in high temperatures at or in the vicinity of the strain gauges. This can result in a drift in sensor output which can skew the results of the stalk strength estimation. In particular, elevated temperatures at or around the strain gauge can cause the sensor output to be higher than intended, causing the stalk strength to be over-estimated (that is, a weaker stalk may present as a stronger stalk) Accordingly, temperature sensor 92 is mounted to the housing in the vicinity of the strain gauges in areas of the harvester that can experience change in temperature during harvester operation such as huge field which can take hours for harvest. With a statistically experimental setup to harvest same hybrid at varied location in same field at different time point in day can lead to harvesting at different temperature due to heat generated by continuous operation and ambient temperature, this sensor configuration can help capture response to increase in temperature which can be characterized. In one example embodiment, the temperature sensor 92 is mounted at base of the housing 50 to study temperature variation in different row of harvester. Temperature sensor 92 may be, as non-limiting examples, a thermocouple, a resistance temperature detector (RTD), a thermistor, a semiconductor-based integrated circuit thermocouple, or any other sensor configured to measure, infer, or estimate temperature.

Additionally, or optionally, in further embodiments, a geopositioned sensor 94, or other GPS device, is mounted to the housing 50. This enables positional information regarding the plants on the plot being harvested to be known, such as the identity of a plant in the plot (plant reference or identity number, plant background, etc.). In alternate embodiments, in lieu of a sensor, the controller receiving input from the strain gauges may be communicatively coupled (e.g., via wireless communication) to an alternate source of GPS information regarding the position of the plants in the plot. By overlaying the output of such a map with the output of the sensors, a correlation may be made as to the stalk strength of each plant being harvested on the plot. In this way, a breeder may be able to accurately estimate and assess the stalk strength of each plant on the plot. In addition, even if a plurality of plants of varying background, breeding line, or trait combinations are grown on a common plot, a controller may be able to compute an average stalk strength of each plant variety by overlaying the output of the sensors with a plot map.

In the depicted embodiment, the two strain gauges 56 (herein also referred to as strain sensors 56a, 56b) are located on a lateral side of each of the leg sections 52, 54 (FIG. 8). Since the force exerted by the stalks against crushing by the rolls 40, 42 tends to increase the separation of the leg sections 52, 54, strain gauges 56 measures cross-sectional force. In another embodiment two strain gauges are located at the bottom of the base 50 measures compression effect at the base. The lead wires 58 of the strain gauges 56 are wired as in the circuit 62 of the diagram of FIG. 15. The output signals of the circuit 62 are connected to a Signal Amplifer 66 configured to transform the output signals of the strain gauges to amplified and scalable analog signals to 24-bit digital output. Any known Signal Amplifier may be used that provides the requisite signal amplification. As non-limiting examples, the signal amplifier may be VNR Industries Amplifier Model No. X67AI2744 or X90CP174.24. The amplifier also enables the sensors to be coupled to a display (such as a display of the controller or a dedicated control unit) for displaying the amplified output of the sensors to an operator. The signal is then (or thereby) relayed to a controller configured with computer-readable instructions for generating an output indicative of the stalk strength of the stalk crushed through the rolls based on the input received from the one or more strain gauges 56. In one example embodiment, the controller is an on-board controller electrically and communicatively coupled to the strain gauges and temperature sensors. For example, the controller may be coupled in a common electrical box with the strain gauge amplifiers and a power supply unit providing electrical power to the sensors. Optionally, the on-board controller is coupled to a display for displaying sensor readings and/or a user interface for receiving user input (such as a dial or knob interface via which an operator can indicate a degree of sensor signal amplification desired).

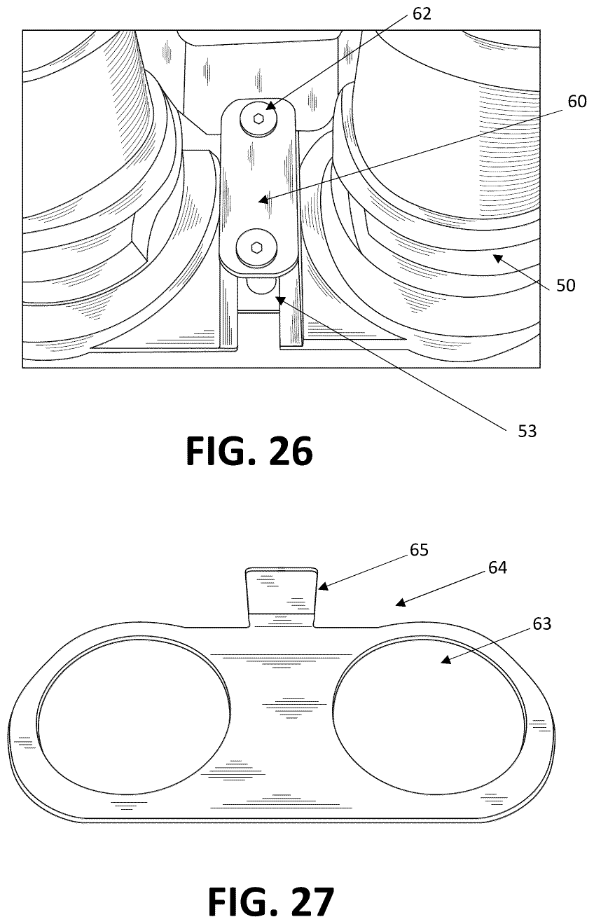

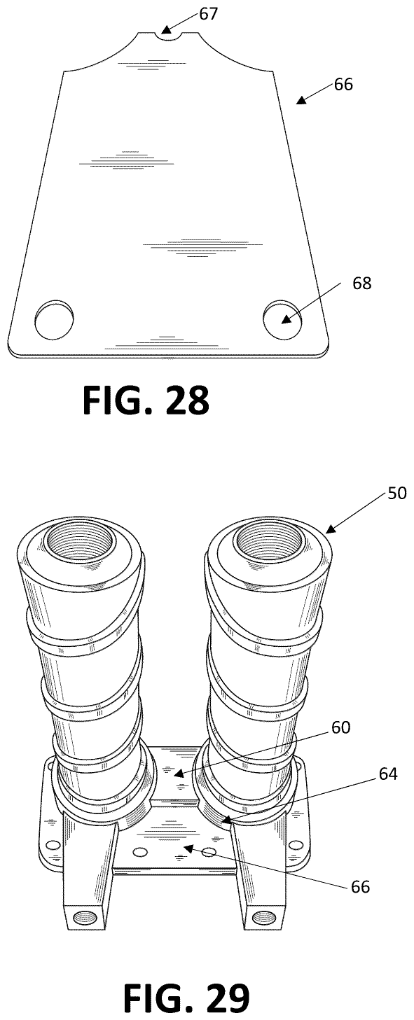

In embodiments, one or more of the sensors (e.g., one or more or all of the temperature sensors and/or the strain gauge sensors) may comprise a protection assembly as shown in FIGS. 26-29. The protection assembly comprises a sensor protection cover as shown in FIG. 26, a protection plate as show in FIG. 27 and a protective cable cover as shown in FIG. 28. F (G. 29 shows the drive shaft housing with all components of the sensor protection cover mounted. Since the strain gauge sensor has a small surface area and electrical contact, the location of the sensor, when mounted, exposes it to heavy impact from corn stalks hitting the base of the bracket. The sensor protection cover 60, when mounted to the housing using mounting screws 62, shields the underlying strain gauge sensors. In the embodiment depicted at FIG. 26, the strain gauge sensors are mounted in the inter-leg region 53 of the housing 50 and the protection cover accordingly is also mounted in the base of the housing, in the inter-leg region. The protective covering may be made of any suitable metal, such as stainless steel, aluminum or galvanized steel, and provided to cover the underlying sensor and sensor contact pads and connector cable and provide protection from environmental conditions. The protection thickness may be in the order of millimeters so as to give sufficient clearance between the bracket mounting and crushing blades of the stalk crusher. The sensor protective covering is securely attached to the stalk crusher using any suitable attachment, such as a mounting screw 62 in the depicted embodiment, so to act as primary protection. The sensor base plate protection is secured tightly between the bracket or protection plate 64 (FIG. 27) and crushing blades. Protection plate 64 has a raised stopping surface 65 and cut outs 63 designed to allow the plate to be positioned over the base of the housing. Cable protection cover 66 (FIG. 28) is mounted the base region 57 of the housing using the screws through mounting holes 68 of the cover. A notch 67 on the cover 66 allows for improved alignment with the base region of the housing. Cable protection cover 66 covers the terminal block 60 and protects any underlying cables.

An example embodiment of the coupling of the sensors to the controller is shown at the block diagram of FIG. 23. Each strain gauge 56 is connected to a strain gauge amplifier which feeds the 24-bit digital signal to on-board controller. The triggering of data acquisition is controlled in the cabin by the press of a button by the operator. In one embodiment, at the start of every plot, the operator pushes the trigger button which triggers harvesting operation as well as sends the current Range and Row information of the plot to the on-board Controller from the Harvester Software named Harvest Master. In the same way, at the start of the immediately next plot, the operator pushes the trigger button again which stops the data acquisition from the earlier plot (e.g., a first plot) and starts data acquisition for the next plot (e.g., a second plot immediately after the first plot). Thus, each button press defines the single point at which one plot ends and another plot begins. Pressing of the button at the onset of each plot triggers the controller to compute the aggregate stalk strength force for the entire plot it has just completed collecting data on. This information is then later pushed to internal databases, such as via a plugin (e.g., FieldAERO plugin) in a master controller software (e.g., Harvest Master software). Thus, with reference to the earlier example, pressing of the button at the beginning of the second plot triggers termination of data collection for the first plot, aggregate stalk strength estimation for the first plot, and initiation of data collection for the second plot.

In an alternate embodiment, at start of every plot operator pushes the trigger button which triggers harvesting operation as well as sends the current Range and Row information of the plot to the on-board Controller from the Harvester Software named Harvest Master. At the end of plot, the Harvest Master sends a stop signal to the controller to stop the data acquisition and it computes and aggregate stalk strength force for entire plot. In some embodiments, the operator pushes the trigger button again which stops the data acquisition and sends signal to controller to stop the data acquisition and it computes and aggregate stalk strength force for entire plot. This information is then later pushed to internal databases, such as via a plugin (e.g., FieldAERO plugin) in a master controller software (e.g., Harvest Master software).

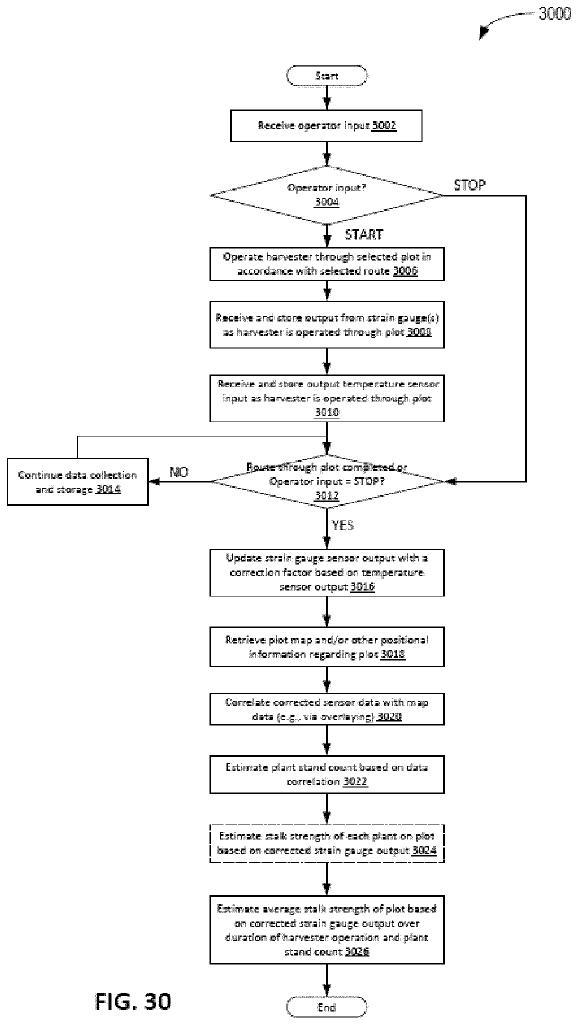

Alternatively, the stalk strength estimating controller may be electrically and communicatively coupled to a controller of the harvester (such as a main controller of a computer in an operator cabin of the harvester, the main controller optionally coupled to a display and a user interface for receiving operator inputs). In still further embodiments, the stalk strength estimating controller coupled to the electrical box is a control module of the main harvester controller. In yet further embodiments, the stalk strength estimating controller is an off-board controller located at a remote location and communicatively coupled, such as via wireless communication, internet, cloud services, etc., to the harvester controller on-board the harvester. In all embodiments, the controller is configured to receive a raw signal from the strain sensor(s) and digitally process the signal for computing a force value indicative of a stalk strength of the harvested plant. Further, the controller is configured to process the signals to compute a number of plants harvested and provide a numerical value indicative of the average stalk strength of the harvested plot. This computed Force Value is stored in a database as a function of a position within the plot (e.g., a Range and Column of that plot). An example embodiment of a method performed by the controller for assigning a stalk strength estimate value to a plot is detailed below at FIG. 30.

A communication interface, such as a M12 Twisted Shielded cable, can be used to connect the strain gauge sensors to the data acquisition module. It may be further interfaced to an on-board controller using X2X Link cable for processing and analysis of the digital signals. In other examples the communication interface may be a wired or wireless interface. A data storage unit is attached to a laptop for storage of data received from the one or more strain sensors and temperature sensors.

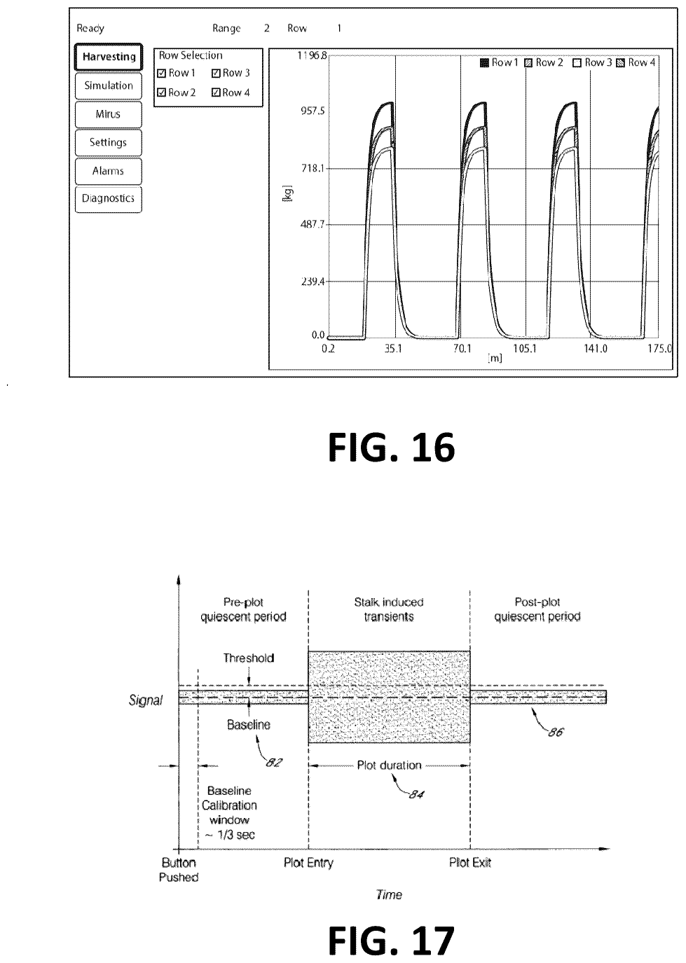

Software on the controller 70 includes a graphical user interface (GUI) with one or more controls that are configured to receive input from an operator and provide an output to a display in accordance with the input. As non-limiting examples, the GUI is configured to provide a “HARVEST” screen for displaying and receiving input on a plurality of parameters related to the operation of a harvester for a harvesting operation, a “SIMULATION” screen for displaying and receiving input on a plurality of parameters related to the simulation of a combine in harvesting operation, and a “SETTINGS” screen for setting acquisition rate of various sensors and units of sensor readings. A sample “HARVEST” screen is shown in FIG. 16. The signal generated by the strain gauges 56 is displayed on the graphical user interface 78, an example of which is shown in FIG. 21. The controls on the HARVEST screen 78 displays option to cycle sensors readings/graph of different rows. The data acquisition period is controlled by trigger button in cabin and the position and or velocity sensor of the combine. Every assertion of the HMRE switch causes the combine position to advance to the next range/row coordinate as defined in the COMBINE screen. When a scan is complete, the collected data is displayed along with the minimum and maximum collected values. This allows the operator to discern data collection parameters such as if the collected data has a well-behaved shape.

As used herein, initiation of data collection comprises the controller actuating the strain gauge sensors (e.g., by initiating power delivery to the sensors), receiving sensor input, and storing the sensor data in a buffer computing digital signal processing in real-time at the controller and then saving it to database (e.g., as a function of plant identity or plot position).

The harvest boundary defines a rectangular region using range and row coordinates in the specified location/field. The sampling information is used to configure the data acquisition device. Specifically, the Freq is used to set the sampling frequency and the duration defines that maximum length of time in seconds that the data acquisition device will collect data. Alternatively, the duration refers to the amount of time data was collected and stored in a buffering file before closing the file and opening another buffering file. The Rng defines the direction the combine is moving in the field. This can be set to “A” for range ascending or “D” for range descending. The Row defines the direction the combine is moving in the field. This can be set to “A” for row ascending or “D” for row descending.

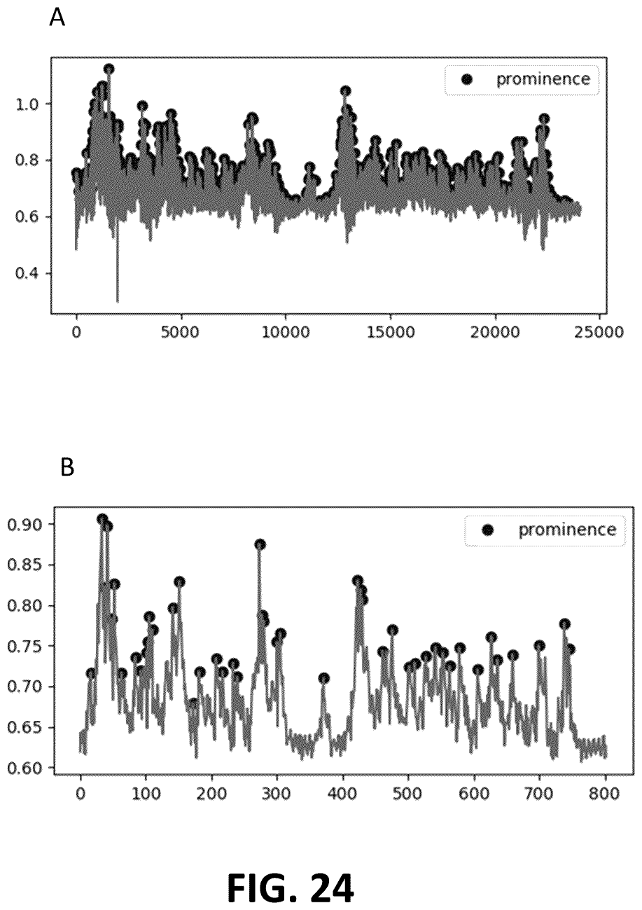

The inventors herein have recognized that by adjusting the sampling frequency of the data acquisition device (comprising the strain gauge sensors), a plant stand count can be provided which enables a more accurate estimation of the average stalk strength of a plot. In one example, the inventors found that reducing the sampling frequency from 3000 Hz (FIG. 24A) to 100 Hz (FIG. 24B) resulted in an improved resolution of sensor output peaks. By overlaying these output peaks with a map of the position of plants on the plot from a precision planter, a controller may be configured to correctly estimate the number of plants that were standing at the time of harvest, thereby providing a plant stand count. This number can then be used to calculate the average stalk strength of plants on the given plot and distribution of stalk strength within a plot to estimate strength variation at start, mid, versus end of a plot. An example of this is shown at FIG. 24.

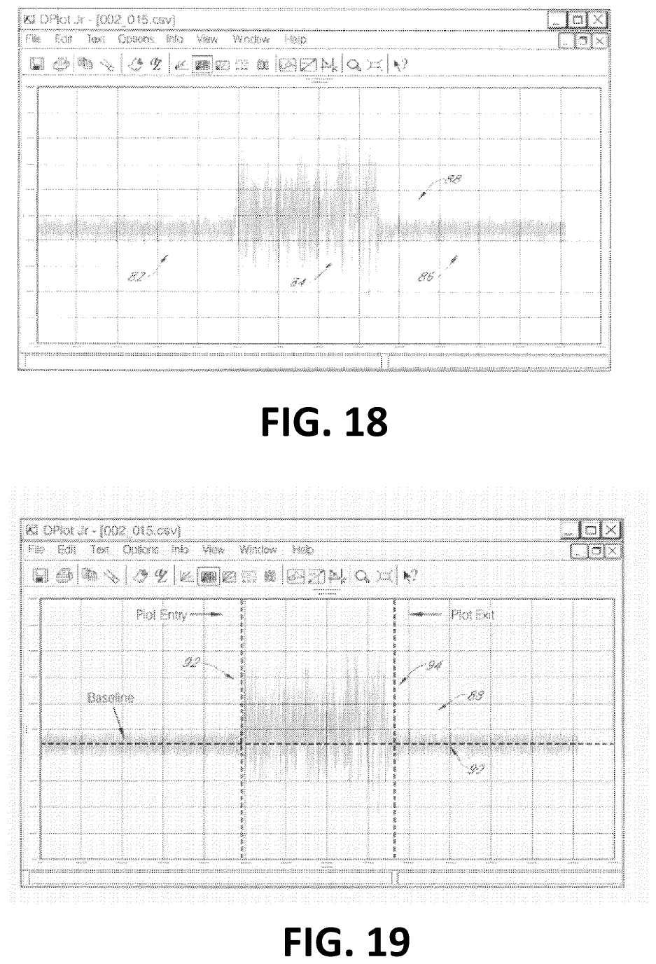

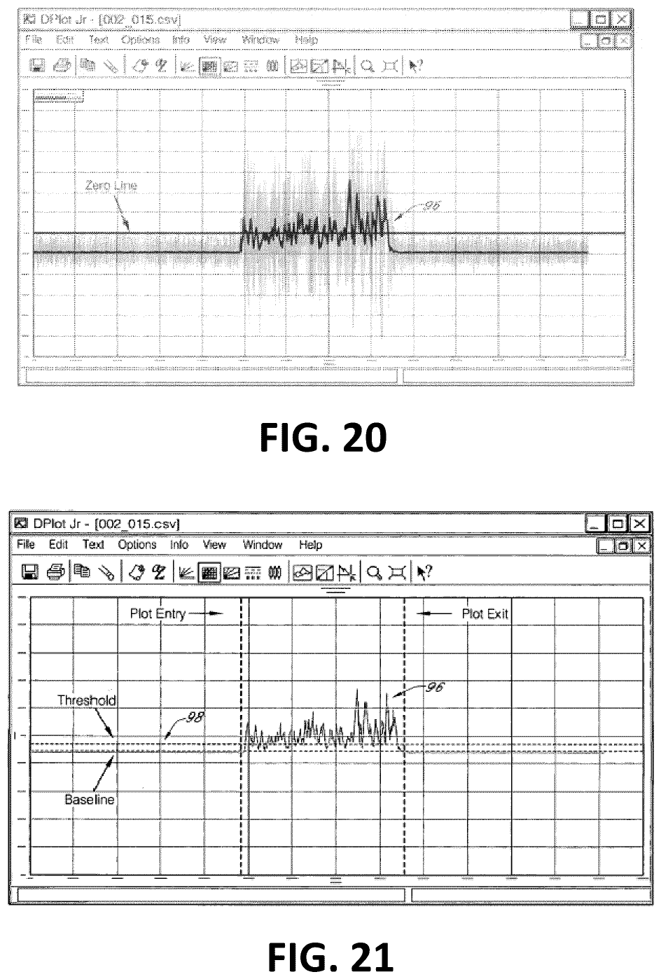

A signal for processing by the software having ideal characteristics is illustrated in FIG. 17 The signal includes a pre-plot quiescent period 82, a period of stalk-induced transients 84, and a post-plot quiescent period 86. The period of stalk-induced transients 84 begins at entry of the harvester 30 into the plot and ends at exit of the harvester 30 from the plot. An example of a data signal 88 generated during the pre-plot quiescent period 82, a period of stalk-induced transients 84, and a post-plot quiescent period 86 is shown in FIG. 18 which is another of the display screens that may be selected from the graphical user interface 78. The baseline 90 and the plot entry 92 and plot exit 94 times of the ideal signal are shown in FIG. 19. The signal is passed through a software algorithm that applies a low pass finite impulse response (FIR) digital filter (5 Hz; 1000 taps) to the raw data signal, generating the filtered signal 96 shown in dark line in FIG. 21. The software also takes an average of the raw data signal every 1000 samples.

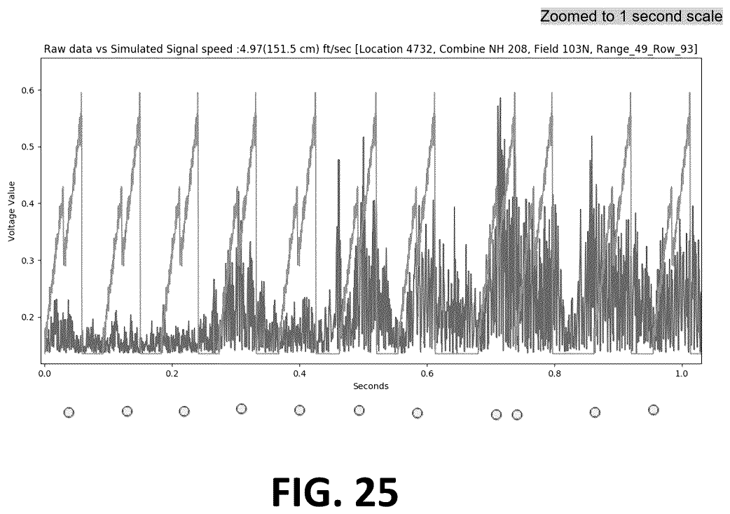

As observed in FIG. 25 the prominent peaks may be correlated with the plant being crushed by the harvester when data is captured at a sampling frequency of 3000 Hz. The peaks are saturated and overlapped more than number of plants present in a plot thus adding noise due to oversampling. Whereas when sampling frequency was brought down to 100 Hz, prominent peaks in signal can be observed with a correlation with plant stand in that plot.

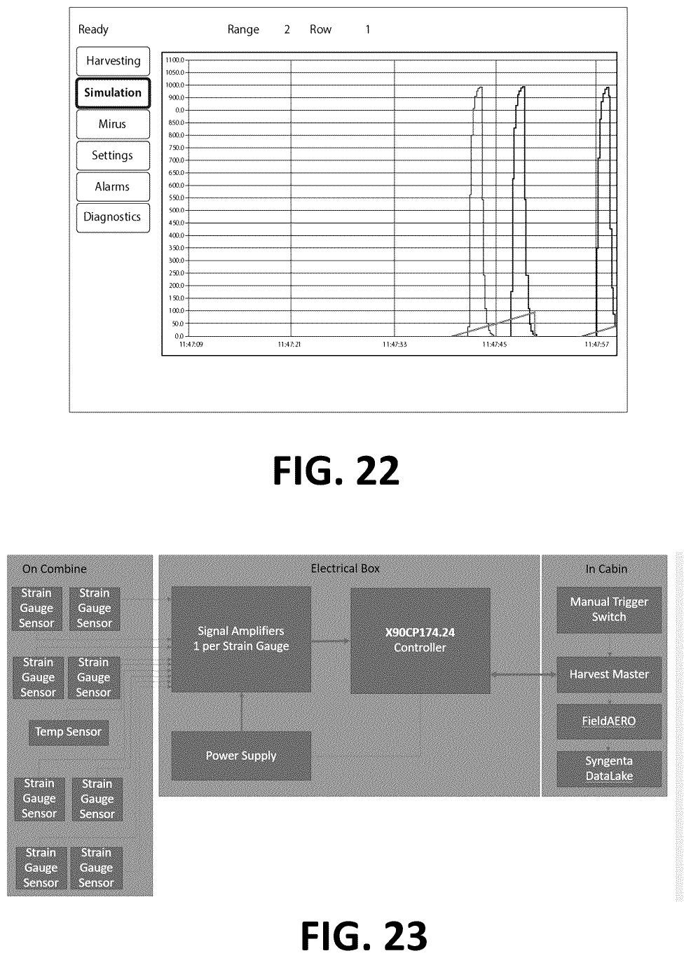

The filtered signal 96 is displayed by itself in FIG. 22. The software allows a user to set a threshold 98 below which no data will be taken. The threshold 98 is set above the baseline by an amount that is above most of the baseline noise, but which also is below most of the data in the filtered signal. Where the threshold 98 crosses the filtered signal on the left is defined as “plot entry” and where it crosses on the right is defined as “plot exit”. Only data between “plot entry” and “plot exit” and above the threshold 98 is collected and analyzed.

An example embodiment of a method 3000 performed by a controller for assigning a stalk strength estimate value to a plot through which a harvester has been operated is detailed at FIG. 30.

The method comprises, at 3002, receiving operator input. For example, an operator operating the harvester (e.g., while in a harvester operator cabin) may provide input to the controller (e.g., an on-board control unit coupled to a display in cabin) via an interface (e.g., keyboard, touchscreen, mouse, stylus or other input device). In one example, operator input is provided by the operator engaging or actuating a “Start” button. Other operator inputs include details regarding the plot to be harvested (such as coordinates and boundary of plot, plot map, etc.) and harvesting parameters (such as a planned route of harvesting, a speed of harvester operation, sensor sampling frequency, etc.).

In response to the operator input comprising “Start” (3004) at 3006 the method includes operating the harvester through the selected plot in accordance with the selected route and other route parameters. At 3008, while operating the harvester, strain gauge sensor input is received and stored. That is, while operating the harvester and receiving and crushing stalks through the stalk rollers, strain gauge sensor inputs are received and stored in a database or memory. Similarly, at 3010, temperature sensor input is received indicative of the temperature absolute at the strain gauges during the plant stalk crushing operation of the harvester and stalk rollers.

At 3012, it is determined if the operator has provided input indicating harvester operation is to be stopped (such as due to completion of the harvesting route). Alternatively, the controller may determine, based on operator input, that the current plot has been completed and a subsequent plot has been started (such as in response to the operator pressing a button at the onset of each plot, wherein the actuation of the button indicates that operation through a first plot has been completed and operation through a second, immediately subsequent plot is being initiated). In some embodiments, the controller may automatically determine that the route is complete based on a duration of harvester operation having elapsed since the start button was actuated. Further still, route completion (e.g., plot completion) may be based on positional information. If the route is not complete, at 3014 the harvester continues to move through the plot, harvesting plant stalks, and strain gauge data continues to be received and stored. If the route is completed, or a “stop” is indicated by the user, then data collection is discontinued, and all retrieved data is stored in the controller's memory. In some embodiments, the collected sensor data is additional or optionally stored in an off-site database or server (e.g., cloud-based server). The data is now ready for further processing. At 3016, the controller updates the collected strain gauge data with the temperature sensor data. For example, the controller may determine a correction factor based on the temperature sensor data (e.g., based on the temperature sensor output at the beginning of the harvester operation relative to at the end if the operation, or based on the temperature sensor output relative to ambient conditions as measured, retrieved, or inferred, such as from a weather database). The controller may then apply the correction factor to the strain gauge output to calculate an updated or corrected strain gauge output.

At 3018, the controller retrieves positional information regarding the plot, such as a plot map. In alternate embodiments, positional information may be continuously retrieved during the harvesting operation, such as from a GPS device or positional sensor coupled to the harvester. At 3020, the corrected strain gauge data is correlated with the positional information, such as by overlaying the plot map with the strain gauge data. At 3022, a plant stand count is estimated based on the correlation. In one example, the correlation includes, identifying sensor peaks, and correlating the maxima of each peak with the plot map to associate each peak with the position of a plant in the plot. As illustrated in FIG. 33 at raw signal at lower frequency is overlaid with a simulated signal which represent stalk crushing peaks. When overlaid with plant position it can be seen the prominent peaks of raw signal overlaps with simulated signal peaks as well the plant position. Thus, through a machine learning algorithm can be applied to an extensive dataset to predict the plant number from the peaks.

At 3024, the method optionally comprises estimating the stalk strength of each harvested plant of the plot based on the corrected strain gauge output. At 3026, the method comprises estimating an average stalk strength of plants harvested across the plot based on the corrected strain gauge output and the plant stand count. For example, based on the corrected strain gauge output collected over the duration of harvester operation, and the total number of plants harvested over that duration, an average stalk strength value is assigned to the plot. These stalk strength estimates (individually for each plant or average across all plants of a plot) can be used to select plants for use in breeding programs.

In this way, the present invention gathers data representative of a number of plants harvested from a particular plot, the stalk strength of each harvested plant, and the average stalk strength of corn plants harvested from the particular plot and stores that data for subsequent analysis and use. For example, the present invention is particularly useful in corn hybrid breeding programs where breeding decisions may be based, at least in part, on the stalk strength of particular experimental or research hybrids under consideration. For example, plants having a higher than threshold stalk strength (or plants from plots having a higher than threshold stalk strength value) may be selected for use as a breeding partner while plants having a lower than threshold stalk strength (or plants from plots having a lower than threshold stalk strength value) may be limited for use in breeding programs.

The present invention can be used to detect plants in the rows of a plot being harvested by correlating sensor peak signals, received while operating at a reduced sampling frequency, with plot maps, to identify a number, and optionally an identity, of plant stalks being crushed over a period of time. By using the present invention with a GPS device whereby the data being collected is associated with a geographical location, the location of the harvested plant within the plot can be determined. A reliable estimation of the number of plants harvested on a plot increases the accuracy of average stalk strength estimation for a given plot.

The present invention may also be used to detect gaps of plants in the rows of a plot being harvested when there is no signal indicative of plant stalks being crushed for a period of time. By using the present invention with a GPS device whereby the data being collected is associated with a geographical location, the length and location of the gap can be determined. Another application of the present invention when used with a GPS device is the calculation of a “fill ratio” which represents how uniformly the plants are distributed in a plot being harvested.

Non-limiting embodiments of the invention comprise apparatus for measuring a stalk strength of a plant growing in a plot as well as methods of using such apparatus for measuring the stalk strength of plants used in breeding.

One example embodiment of an apparatus for measuring stalk strength of a plant growing in a plot comprises a roller that is rotated to engage and crush a stalk of the plant; a force sensor coupled to the roller for measuring a force exerted on the roller by the plant stalk in resistance to crushing by the stalk roll; and a temperature sensor coupled to the roller for measuring a temperature at or around the force sensor. In embodiments, the temperature sensor is configured to measure an ambient temperature at or around the force sensor. In particular embodiments, the temperature sensor is configured to measure a change in the ambient temperature at or around the force sensor over a duration of apparatus operation. In embodiments, the roller is driven by a shaft, and wherein one or more of the force sensor and the temperature sensor is coupled to a housing of the shaft. In embodiments, the apparatus further comprises a controller configured with computer readable instructions stored in a memory for: receiving a signal from the force sensor; receiving another signal from the temperature sensor; and correcting the force sensor signal based on the temperature sensor signal. In embodiments, the controller is configured with further instructions for receiving data indicative of a position of the plant within the plot; and correlating the corrected force sensor signal with the position of the plant within the plot. In embodiments, the controller is configured with further instructions for receiving data indicative of a position of the plot; and correlating the corrected force sensor signal with the position of the plot. In embodiments, the controller is configured with instructions for correlating a statistical average of the corrected force sensor signal for a threshold number of plants with the position of the plot, wherein the threshold number of plants are growing in the same plot, and wherein the statistical average includes one of a mean, mode, median, or weighted average. In embodiments, the method further comprises a geopositioned sensor for generating the data indicative of the position of the plot and/or the position of the plant within the plot, the geopositioned sensor optionally coupled to a housing of the roller. In embodiments, the controller is communicatively coupled to a database, and wherein the controller includes further instructions for retrieving a map indicative of the plot and/or the position of each plant within the plot; and storing the signal received from the force sensor as a function of the position of the corresponding plant in the plot. In embodiments, the controller is configured with further instructions for assigning a stalk strength value to the plant and/or the plot as a function of the corrected force sensor signal. In particular embodiments, the plant is a corn plant.

In further embodiments, the apparatus further comprises a protection assembly mounted to the base of the drive shaft housing for protecting the force sensor. In particular embodiments, the protection assembly comprises a sensor protection cover mounted over the force sensor at the base of the drive shaft housing; and a protection plate mounted over the sensor protection cover. In still further embodiments, the protection assembly further comprises a protective cable cover coupled to the protection plate and extending over the base region of the housing to protect underlying cable wires coupling the force sensor to the controller.

Non-limiting embodiments of a method of selecting corn plants with enhanced stalk strength, the method comprising the steps of while operating harvesting equipment through a plot of corn plants, crushing a stalk of a corn plant received at the harvesting equipment; measuring, via a force sensor coupled to the harvesting equipment, a force exerted by the stalk of said plant against the stalk crushing; measuring, via a temperature sensor coupled to the harvesting equipment, a temperature parameter at the force sensor during the harvesting; estimating plant stalk strength as a function of an output of each of the force sensor and the temperature sensor; and selecting corn plants of the plot with a higher than threshold plant stalk strength for use in a breeding program. In embodiments, the force sensor measures a force exerted by the stalk of each plant of the plot against the stalk crushing as the harvesting equipment operates through the plot. In embodiments, the force sensor measures a force exerted by the stalk of a threshold number of plants of the plot against the stalk crushing as the harvesting equipment operates through the plot. In embodiments, the estimating comprises estimating an average plant stalk strength value for plants of the plot based on a statistical average of the force measured by the force sensor. In embodiments, the harvesting equipment comprises one or more stalk crushing rolls coupled to a harvester, and wherein the force sensor is a strain gauge coupled to a housing of the rolls. In embodiments, the estimating comprises estimating a force sensor correction factor based on the output of the temperature sensor; correcting the output of the force sensor with the correction factor; and calculating a stalk strength value for one or more or each plant of the plot based on the corrected force sensor output. In embodiments, the method further comprises receiving, during the harvesting, positional information for the plot and/or each plant within the plot; correlating the corrected force sensor output with the positional information; and estimating a number of plants harvested during the harvesting based on the correlation. In embodiments, the positional information is received from a geopositioned sensor coupled to a housing of the harvesting equipment or inferred from a plot map retrieved from a database. In embodiments, the method further comprises storing the force sensor output as a function of the positional information.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

1. An apparatus for measuring stalk strength of a plant growing in a plot, comprising:
a roller that is rotated to engage and crush a stalk of the plant;
a force sensor coupled to the roller for measuring a force exerted on the roller by the plant stalk in resistance to crushing by the stalk roll; and
a temperature sensor coupled to the roller for measuring a temperature at or around the force sensor.

2. The apparatus of claim 1, wherein the temperature sensor is configured to measure an ambient temperature at or around the force sensor.

3. The apparatus of claim 2, wherein the temperature sensor is configured to measure a change in the ambient temperature at or around the force sensor over a duration of apparatus operation.

4. The apparatus of claim 4, wherein the roller is driven by a shaft, and wherein one or more of the force sensor and the temperature sensor is coupled to a housing of the shaft.

5. The apparatus of any of claims 1-4, further comprising a controller configured with computer readable instructions stored in a memory for:
receiving a signal from the force sensor;
receiving another signal from the temperature sensor; and
correcting the force sensor signal based on the temperature sensor signal.

6. The apparatus of claim 5, further comprising a geopositioned sensor for generating the data indicative of the position of the plot and/or the position of the plant within the plot, the geopositioned sensor coupled to a housing of the roller, wherein the controller is configured with further instructions for:
receiving data indicative of the position of plot and/or the position of the plant within the plot from the geopositioned sensor; and
correlating the corrected force sensor signal with the position of the plot and/or the position of the plant within the plot.

7. The apparatus of claim 6, wherein the controller is further configured with instructions for:
correlating a statistical average of the corrected force sensor signal for a threshold number of plants with the position of the plot, wherein the threshold number of plants are growing in the same plot, and wherein the statistical average includes one of a mean, mode, median, or weighted average.

8. The apparatus of claim 6, wherein the controller is communicatively coupled to a database, and wherein the controller includes further instructions for:
retrieving a map indicative of the plot and/or the position of each plant within the plot; and
storing the signal received from the force sensor as a function of the position of the corresponding plant in the plot.

9. The apparatus of any of claims 5-8, wherein the controller is configured with further instructions for:
assigning a stalk strength value to the plant and/or the plot as a function of the corrected force sensor signal.

10-11. (canceled)

12. The apparatus of claim 11, further comprising a protection assembly mounted to the base of the drive shaft housing for protecting the force sensor, the protection assembly comprising:
(a) a sensor protection cover mounted over the force sensor at the base of the drive shaft housing;
(b) a protection plate mounted over the sensor protection cover; and
(c) a protective cable cover coupled to the protection plate and extending over the base region of the housing to protect underlying cable wires coupling the force sensor to the controller.

13. (canceled)

14. The apparatus of claim 1, comprising at least two force sensors mounted in a biaxial configuration to the drive shaft housing, and optionally wherein the plant is a corn plant.

15. A method of selecting corn plants with enhanced stalk strength, comprising the steps of:
while operating harvesting equipment through a plot of corn plants, crushing a stalk of a corn plant received at the harvesting equipment;
measuring, via a force sensor coupled to the harvesting equipment, a force exerted by the stalk of said plant against the stalk crushing;
measuring, via a temperature sensor coupled to the harvesting equipment, a temperature parameter at the force sensor during the harvesting;
estimating plant stalk strength as a function of an output of each of the force sensor and the temperature sensor; and
selecting corn plants of the plot with a higher than threshold plant stalk strength for use in a breeding program.

16. The method of claim 15, wherein the force sensor measures a force exerted by the stalk of each plant of the plot against the stalk crushing as the harvesting equipment operates through the plot, wherein the harvesting equipment comprises one or more stalk crushing rolls coupled to a harvester, and wherein the force sensor is a strain gauge coupled to a housing of the rolls.

17. The method of claim 15, wherein the force sensor measures a force exerted by the stalk of a threshold number of plants of the plot against the stalk crushing as the harvesting equipment operates through the plot.

18. The method of claim 17, wherein the estimating comprises estimating an average plant stalk strength value for plants of the plot based on a statistical average of the force measured by the force sensor.

19. (canceled)

20. The method of claim 15, wherein the estimating comprises:
estimating a force sensor correction factor based on the output of the temperature sensor;
correcting the output of the force sensor with the correction factor; and
calculating a stalk strength value for one or more or each plant of the plot based on the corrected force sensor output.

21. The method of any of claim 15-18 or 20, further comprising:
receiving, during the harvesting, positional information for the plot and/or each plant within the plot;
correlating the corrected force sensor output with the positional information; and
estimating a number of plants harvested during the harvesting based on the correlation.

22. The method of claim 21, wherein the positional information is received from a geopositioned sensor coupled to a housing of the harvesting equipment or inferred from a plot map retrieved from a database, and wherein the method further comprises storing the force sensor output as a function of the positional information.

23. (canceled)

24. The method of claim 15, wherein measuring the force comprises measuring an output of the force sensor coupled to a base of a driving shaft housing of one or more stalk crushing rollers of the harvesting equipment.

25. The method of claim 15, wherein measuring the force comprises measuring an output of at least two force sensors mounted in a biaxial configuration to a driving shaft housing of one or more stalk crushing rollers of the harvesting equipment.
